(19) United States
(12) Patent Application Publication
(10) Pub. No.: US 2009/0163599 A1
(43) Pub. Date: Jun. 25, 2009

(54) Mif adsorbant

(76) Inventors: Markus Storr, Filderstadt (DE); Reinhold Deppisch, Hechingen (DE); Werner Beck, Rottenburg (DE); Jürgen Bernhagen, Aachen (DE); Ina Merz, Waiblingen (DE); Georg Geiger, Böblingen (DE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/596,534

(22) PCT Filed: Jun. 3, 2005
(86) PCT No.: PCT/EP2005/052572

§ 371 Date: Nov. 24, 2008

Related U.S. Application Data
(60) Provisional application No. 60/582,386, filed on Jun. 22, 2004.

(30) Foreign Application Priority Data
Jun. 18, 2004 (DE) 10 2004 029 573.5

(51) Int. Cl. A61K 31/137; A61P 29/00

(52) U.S. Cl. 514/646

(57) ABSTRACT

The present invention concerns an apheresis material or adsorbant and a method for removing, depleting or inactivating MIF (macrophage migration inhibitory factor) from blood, blood plasma, blood serum or other body fluids. The present invention is also concerned with the use of said apheresis material or adsorbant. In order to prepare a novel means and novel method, which can reduce the activity or amount of the mediator for sepsis and septic shock, MIF, in a patient's body fluid in a manner which is more pleasant and tolerable for the patient than prior art means and methods, the invention proposes that the apheresis material or adsorbant comprises a solid carrier material on the surface of which MIF-binding molecules or functional groups are immobilized. The method proposes that the apheresis material or adsorbant be brought into contact extracorporeally with the blood, blood plasma, blood serum or other body fluids.

### OBJECT OF THE INVENTION

The invention relates to an apheresis material or adsorbant and to a method for removing, depleting or inactivating MIF (macrophage migration inhibitory factor) in blood, blood plasma, blood serum or other body fluids, in particular such body fluids from a patient with sepsis or septic shock. The invention also relates to the use of said apheresis material or adsorbant to remove, deplete or inactivate MIF in blood, blood plasma, blood serum or other body fluids.

### BACKGROUND OF THE INVENTION

### Cytokines

Cytokines are proteins formed by various cells, which influence the behavior of other cells. Many cytokines primarily affect their target cells via specific receptors, usually to trigger cell growth, differentiation or death.

Above all, cytokines play an important role in the inflammatory reaction and its regulation, in which they affect the blood vessels together with other inflammatory mediators. They cause dilation and increased permeabilities of the blood vessels, resulting in an increased blood flow and distribution of fluid in the region of the seed of the infection.

In an inflammatory reaction, cytokines also increase expression of adhesion molecules of the vessel wall endothelium, to recruit immune cells and enable them to move to the seed of the infection. At the same time, cytokines activate immune cells. Thus, cytokines are primarily mediators within the immune system, which carry out various functions and thus regulate inflammatory reactions.

A cytokine can be formed by various types of cells and can also affect various types of cells. Usually, cytokines are synthesized as a response to inflammatory or antigenic stimuli and normally have a local effect (autocrine or paracrine). Some cytokines also have an endocrine effect, in the same manner as hormones. In contrast to hormones, however, they are produced by various cell types.

Cytokines are polypeptides or glycoproteins with a molecular weight of ≦30 kDa and can be classified into various sub-families by their structure, such as hematopoetins, interferons or TNF families.

Since cytokines play an important role in immune reactions, they are often involved in diseases, such as septic shock or sepsis, autoimmune diseases and rheumatoid arthritis. They are mediators in inflammatory reactions and also in sepsis. As an example, they are formed as a reaction to an invading microorganism and trigger the inflammatory reaction by forming further mediators, free radicals, eicosanoids, etc. This cascade of events activates the immune system. Among the various mediators of the sequelae of events that can eventually lead to sepsis, the cytokines TNF, IL-1 and MIF play a pivotal role. Thus, MIF is presumed to be a major mediator of sepsis. Discovery of novel mediators of sepsis and devising therapeutic strategies against them is of importance, as strategies such as the administration of anti-TNF antibodies or antagonists for IL-1 receptors produce no positive results in those afflicted with sepsis.

### Macrophage Migration Inhibitory Factor (MIF)

The cytokine macrophage migration inhibitory factor, MIF, was discovered independently by Bloom & Bennett and by David in 1966 and described as a T cell product which inhibits the migration of macrophages. Later, further properties and effects were discovered for MIF, such as stimulation of the activity of macrophages and control of the immune response, which extends far beyond the function of a T cell cytokine.

Cloning MIF (human MIF) was carried out successfully for the first time in 1989 and opened new opportunities for the characterization of this cytokine. However, the migration inhibitory effect on macrophages and the induction of TNF secretion could only be clearly demonstrated following the production of recombinant MIF (rMIF).

MIF is a ubiquitous protein which is found in nearly all cells. It is a regulator for the innate immune system and the immune response and is released under very different conditions. It also plays a role in the regulation of other cytokines, and in the expression of receptors (for example TLR-4), which are involved in the innate immune system. Further functions are the inhibition of p53 and the modulation of components of mitogen-activated protein kinase (MAP-kinase) and Jun-activation domain binding protein-1 (JAB-1) cellular routes.

MIF plays a central role in the inflammatory cascade but is formed by cells that are outside the immune system, such as cells in the endocrine and nervous systems. Further, it is formed from cells which are stimulated by small doses of glucocorticoids. Normally, glucocorticoids inhibit the expression of cytokines. Clearly, MIF and glucocorticoids operate mutually as antagonists and thus regulate the immune response. MIF is thus responsible for activation of the immune system and, inter alia, increases the expression of pro-inflammatory cytokines such as TNF, IL-1, IL-6 and IL-8 as well as the proliferation of T cells.

Recently, MIF has been linked to many inflammatory diseases such as arthritis, sepsis, anemia, encephalomyelitis, tumor growth etc. For this reason, MIF is more and more frequently being seen as an interesting therapy in inflammatory diseases and in autoimmune diseases. Its catalytic activity offers an important starting point for the development of new MIF inhibitors. It is assumed that the biological activity of MIF resides in its enzymatic reactions, but the connection between the enzymatic and the biological activities of MIF has not yet been explained.

### Structure of MIF

The huMIF gene, which is relatively small (<1000 bp), consists of three exons separated by two small introns (100-200 bp). A 600 bp mRNA transcript has been isolated from various tissues. The consensus sequences, which are possibly involved in the regulation of transcription of the MIF gene, include a cytokine (CK-1) site and a nuclear factor-kB (NF-kB) site, and possibly also a negative glucocorticoid responsive element (nGRE), which has been found in the mouse gene, but not yet in the human MIF (huMIF) gene. Those elements reflect both cytokine activity and also hormone- and glucocorticoid-antagonist functions.

The huMIF monomer comprises 114 amino acids and has a molecular weight of 12.5 kDa. Although potential N-glycosylation sites are available, no N-glycosylation occurs. MIF is also specifically secreted, although a hydrophobic N-terminal signal sequence is not present.

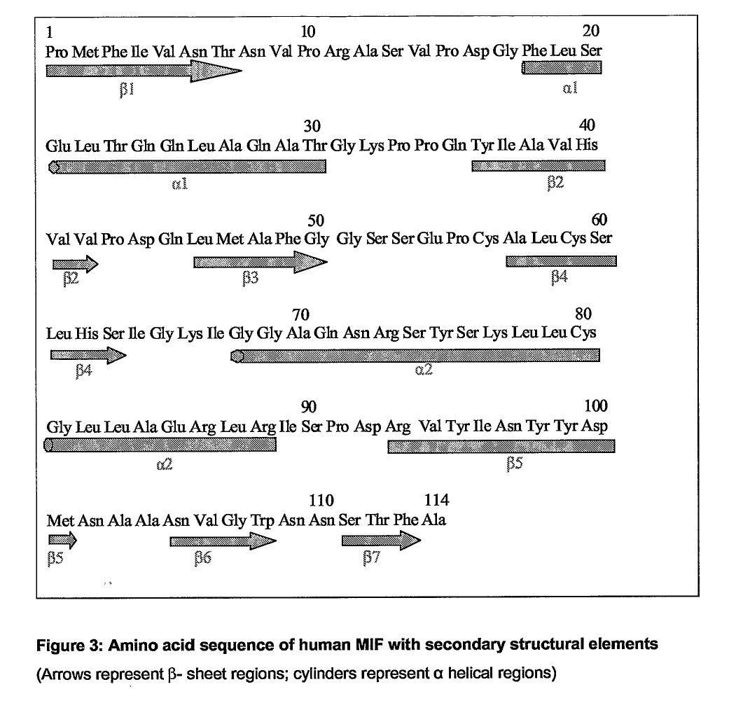

The huMIF monomer consists of two α-helices and six or seven β strands. Four β strands thereof form a central sheet in which two parallel β sheets are bound together anti-parallel. The amino acid sequence of human MIF with secondary structural elements is shown in FIG. 3.

X ray structural analyses show that huMIF is a homotrimer with a size of about 35×50×50 Å, in which three β-sheet regions form a channel structure flanked by six α helices. This structure is unique among all members of the cytokine family. There are also indications that huMIF has a dimeric structure. Some studies indicate that under physiological conditions, a mixture of monomers, dimers and trimers are present. However, the biologically active structure has not yet been elucidated.

### Function and Effect of MIF

The inhibition of macrophage migration by MIF was described very early on. In recent years, further effects caused by MIF have become apparent. MIF is not only released by macrophages but also by T cells and pituitary cells and is thus a cytokine for macrophages and T cells, as well as an endocrine mediator. The difference between the two MIF sources lies among other parameters in the timing of MIF secretion and their dose-effect curves.

A further important role is played by MIF in regulating the immune response, together with glucocorticoids. They are potent anti-inflammatory and immunosuppressive hormones and normally inhibit cytokine production. MIF production, however, is stimulated by small doses of glucocorticoids, and MIF even has a glucocorticoid-overriding activity and reduces its inhibitory effect. An effective immune response after an infection is apparently based on a precisely regulated balance between the anti-inflammatory glucocorticoids and the pro-inflammatory cytokine MIF.

MIF is an important mediator in inflammatory reactions, for example septic shock or sepsis, and in rheumatoid arthritis, in which macrophages contribute to the pathogenesis of the disease.

In addition to immune cells and the cells of the pituitary gland, MIF is also produced by other tissue cells, such as the β-cells of the pancreas. Apparently, MIF plays a role as an autocrine regulator of insulin secretion and may contribute to carbohydrate metabolism.

Elucidating the molecular mechanisms was not possible, primarily because no membrane receptors could be identified for MIF. X ray structural analysis has established homologies with two bacterial isomerases: CHMI (5-carboxymethyl-2-hydroxymuconate isomerase) and 4-OT (4-oxalocrotonate tautomerase). Tautomerase activity has been demonstrated for the non-physiological substrates D-dopachrome and p-hydroxy-phenylpyruvate. These enzymatic binding sites have become focal points for research in the development of MIF inhibitors. They are especially important in the development of new therapeutic strategies in inflammatory diseases, such as sepsis. MIF also plays a role in cellular redox processes.

### Sepsis and Septic Shock

In Western countries, sepsis, septic shock and SIRS (systemic inflammatory response syndrome) are the main causes of death in intensive care units, with death rates between 30% and 70%. In the USA, >500,000 patients per year suffer from sepsis, and the rate is increasing by 1.5% per year.

A problem when treating patients with sepsis is the exact definition of the various stages of sepsis. The following can be distinguished:
a) SIRS systemic inflammatory response syndrome
   temperature >38.3° C. or <36° C.
   raised heartbeat
   raised breathing rate
   increased number of white blood cells
   no microorganisms in blood
b) sepsis systemic reaction to an infection, which is indicated by two or more features of SIRS and microbial infection (SIRS+microbial infection)
c) severe sepsis sepsis which can lead to organ malfunction, hypoperfusion or hypotonia
d) septic shock sepsis-induced hypotonia
e) MODS multiple organ dysfunction syndrome
   severely altered organ function

The spectrum of microorganisms that can initiate sepsis, has changed significantly since the 70s. Initially, gram-negative bacteria were mostly responsible for sepsis; however nowadays, more and more sepsis cases are caused by gram-positive bacteria. A systemic inflammatory reaction as in sepsis can also be triggered by non-infectious stimuli such as trauma, pancreatitis or abdominal and cardiovascular surgery. Thus, it appears that sepsis is triggered by an over-reaction of the immune system. Upon attack by microorganisms, the innate immune system reacts first, whereby neutrophils, macrophages and natural killer cells are mobilized. Here, cytokines play an important role as mediators, which regulate activation and differentiation. Finally, the innate immune system activates the adaptive immune system via these and other stimulating molecules, upon which adaptive immune system has the ability of constructing an immunological memory.

In septic shock, the innate immune system reacts disproportionately strongly, possibly triggering a systemic inflammatory reaction, which eventually leads in the end to organ damage.

### MIF and Sepsis/Septic Shock

Pro-inflammatory cytokines, such as TNF, IL-1 and MIF, play an important role in the development of sepsis. They are mediators which activate the inflammatory reaction and incite the expression of further mediators or the proliferation of inflammatory cells. Compared with glucocorticoids, which inhibit the inflammatory reaction, they are antagonistic and thus strengthen the inflammatory reaction. For this reason, inhibition of this pro-inflammatory cytokine appears to be of interest as a therapeutic strategy in inflammatory diseases and autoimmune diseases.

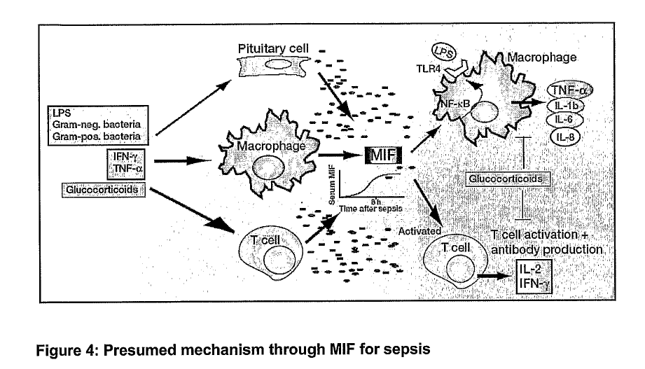

MIF is seen as a major mediator in sepsis, as MIF incites the production of TNF, other pro-inflammatory cytokines and eicosanoids, induces the expression of TLR-4, which recognizes LPS, and activates the innate immune response. MIF and glucocorticoids act as antagonists and are responsible for regulating the inflammatory reaction. MIF has an inhibiting effect on glucocorticoids, which inhibit inflammation. The presumed mechanism through MIF for sepsis is shown in FIG. 4.

In the event of sepsis, the MIF concentration in a patient's serum is substantially increased, whereby the pro-inflammatory reaction is amplified and the prognosis is substantially worsened. Mice lacking the MIF gene, however, are protected from lethal endotoxemia and sepsis occurs only after administering rMIF.

### Therapy Strategies in Sepsis

Pro-inflammatory cytokines constitute an interesting target structure for therapeutic strategies in sepsis, as they trigger and influence occurrence and progress. Until now, therapeutic strategies against TNF and IL-1 have failed in humans, despite successes in animal trials. This has been traced to the fact that therapeutic drugs (antibodies against the corresponding cytokine) in animal trials were administered shortly after injecting LPS, a trigger for bacterial sepsis. A patient with sepsis, however, was much further advanced in the progress of the disease before a clear diagnosis could be made and therapy begun. Other multifactorial reasons and secondary effects were also causes of the negative outcome of those studies in humans.

MIF is seen as a main mediator of sepsis and the first animal trials have produced very promising results. It has been shown that mice with a defective or missing MIF gene have a much higher survival rate after injecting LPS. If MIF is also injected, the death rate in those mice increases. A clear improvement of the survival rate in mice after LPS injection has been shown in other studies with anti-MIF antibodies. Similar results were shown with monoclonal MIF antibodies, and also in animal studies with live bacteria (CLP model).

Various therapy strategies being discussed against sepsis include inhibiting or blocking MIF using specific anti-MIF antibodies. A further strategy is the use of small molecular weight (SMW) inhibitors, which have been developed, for example, so that they inhibit MIF via its enzymatic binding site. MIF is an unusual cytokine in that it appears to exhibit an additional intracellular role that in part is linked to its enzymatic function as tautomerase/oxidoreductase. Blockade of MIF secretion would also constitute a therapeutic strategy. Strategies based on the interaction of MIF with its currently known binding proteins JAB1/CSN5, CD74/Ii, MHCII, BNPL, or myosin light chain kinase (MLCK) may also be considered. In this case, domains or binding moieties for these binding partners could be utilized as MIF neutralizing agents.

### AIM OF THE INVENTION

The aim of the invention is to provide a novel means and new method for reducing the activity or amount of a pivotal mediator of sepsis and septic shock, i.e. the cytokine MIF, in body fluids from a patient in a manner that is more suitable and more comfortable for the patient than current prior art means and methods.

### DESCRIPTION OF THE INVENTION

The invention provides an apheresis material or adsorbant for removing, depleting or inactivating MIF (macrophage migration inhibitory factor) from blood, blood plasma, blood serum or other body fluids. It comprises a solid carrier material with MIF-binding molecules or functional groups immobilized on the surface thereof.

The apheresis material or adsorbant of the invention is particularly suitable for extra-corporal dialysis of blood, blood plasma, blood serum or other body fluids to reduce an excessive MIF concentration or activity and bring it back into the physiological range. In this respect, reduction factors of about 5 to a maximum of about 100 times need to be aimed at. In apheresis, blood from a patient, for example a patient with sepsis or septic shock, is removed from the body either as whole blood or, for example, after separating the blood cells, as blood plasma, and is fed over the apheresis material or adsorbant of the invention. Since the apheresis material or adsorbant of the invention has MIF-binding molecules or functional groups immobilized on its surface, MIF binds to these molecules or functional groups and is removed from the blood or plasma stream or transformed into an inactive form. In addition, the inactivated blood or plasma can be returned to the patient, possibly after further treatment. Because of the reduced MIF concentration or activity, the extent of the inflammatory effect in the body of the patient is reduced and the prognosis improves.

In one embodiment of the apheresis material or adsorbant of the invention, the immobilized MIF-binding molecules or functional groups are bound to the carrier material via a spacer or via polymer chains grafted onto the carrier material.

It has been shown that the binding strength of MIF-binding molecules or functional groups can be considerably improved if they are not directly bound or immobilized onto the surface of the carrier material, but via the spacer or polymer chains grafted onto the carrier material. The improved binding is assumed to be linked to a lower steric hindrance of the MIF bound to the immobilized MIF-binding molecules or functional groups due to the spacing from the surface of the carrier material. A further advantage of the spacer is constituted by multiplication of the MIF binding sites if a plurality of MIF binding molecules or functional groups can be associated with the spacer or polymer chain. This is advantageous if the grafted polymer chains are present to bind the immobilized MIF-binding molecules or functional groups. Production of the material is based on radical graft polymerization of monomers with unsaturated C═C double bonds (for example acrylic acid derivatives) and reactive side chains (for example oxirane groups). Compounds which contain both unsaturated C═C double bonds and oxirane side chains are known to the skilled person. Particular examples are glycidyl methacrylate, glycidyl acrylate and vinyl glycidyl ether. Preferably, glycidyl methacrylate is used. A suitable degree of grafting for use in the invention is in the range 101% to 200%. Preferably, it is between 105% and 120%.

In one embodiment of the invention, the immobilized MIF-binding molecules or functional groups are selected from inhibitors of the catalytic and/or enzymatic activity of MIF. S-hexylglutathione and hexane thiol are known to have a strong inhibiting effect on the dopachrome tautomerase activity of MIF (Swope et al, The Journal of Biological Chemistry, 273: 14877-14884, 1998). Particularly preferably, the immobilized MIF-binding molecules or MIF-binding functional groups are selected from molecules containing mercapto groups or thiol groups. Particularly preferably, in accordance with the invention, mercaptopyridine residues have proved useful as immobilized MIF-binding molecules or functional groups.

In another embodiment of the invention, the immobilized MIF-binding molecules or functional groups are selected from substrates or co-substrates for the catalytic and/or enzymatic activity of MIF. Particularly preferred immobilized MIF-binding molecules or functional groups of this type are selected from catecholamines or derivatives thereof. Experiments have showed that the immobilized catecholamines very effectively bind and deplete MIF from blood and blood plasma. The useful catecholamines are preferably selected from the group consisting of dopa (3,4-dihydroxyphenylamine), dopamine (4-(2-aminoethyl)-benzene-1,2-diol), norepinephrine (noradrenaline; 1-(3,4-dihydroxyphenyl)-2-aminoethanol), epinephrine (adrenaline; 1-(3,4-dihydroxyphenyl)-2-methylaminoethanol) or derivatives thereof. Up to now, the best results to bind and deplete MIF from blood and blood plasma have been achieved using dopamine as the immobilized MIF-binding molecules or functional groups.

More particularly, the apheresis material or adsorbant of the present invention is a porous material, preferably a membrane, a particle bed, a fiber mat or beads. Since the apheresis material or adsorbant of the invention comes into contact with human blood, blood plasma, blood serum or other body fluids, which are subsequently to be returned to the patient, the carrier material is particularly preferably a biocompatible polymeric material. Suitable biocompatible carrier materials are polyethersulfone (PES), polypropylene (PP), polysulfone (PSU), polymethylmethacrylate (PMMA), polycarbonate (PC), polyacrylonitrile (PAN), polyamide (PA), polytetrafluoroethylene (PTFE), cross-linked polystyrene-polyethylene glycol (PS-PEG), cyclo-olefin copolymer (COC), cellulose acetate (CA) or mixtures or copolymers thereof, or mixtures or copolymers with hydrophilized polymers, such as polyvinylpyrrolidone (PVP) or polyethylene oxide (PEO).

In accordance with a further embodiment of the apheresis material or adsorbant, the immobilized MIF-binding molecules or MIF-binding functional groups are selected from anti-MIF antibodies or fragments or derivatives thereof having at least one MIF-specific binding site. Said antibodies, which are specifically directed against MIF, have already been produced or can be produced by the skilled person using known methods, selecting appropriate MIF epitopes. Monoclonal and polyclonal anti-MIF antibodies, preferably monoclonal antibodies, are preferred.

In accordance with a further embodiment of the apheresis material or adsorbant, the immobilized MIF-binding molecules or MIF-binding functional groups are selected from cellular MIF-binding proteins. The skilled person would know of the intracellular protein JAB1/CSN5, a transcriptional co-activator and cell cycle regulator, as well as other suitable subunits of the CSN signalosome complex, the membrane and MHC-associated CD74/invariant chain (Ii chain) protein, the myosin light chain kinase MLCK, and the apoptosis-regulating protein BNPL. Soluble, available, MIF-binding domains or sequences can be identified in these MIF-binding proteins, which can advantageously be employed when immobilized on the adsorbant of the invention.

In a further embodiment of the apheresis material or adsorbant of the invention, the immobilized MIF-binding molecules or MIF-binding functional groups are selected from further inhibitors and substrates for the catalytic and/or enzymatic activities of MIF. Substrates for the tautomerase/isomerase activity of MIF such as dopachrome, phenyl pyruvate or the many inhibitors of said catalytic activity and derivatives of these compounds described to date are suitable. Substrates/co-substrates for the thiol protein oxidoreductase (TPOR) activity of MIF such as glutathione, lipoic acid, hydroxyethyldisulfide, cysteine and other cysteine-containing peptides, such as insulin peptide sequences, are also suitable.

The invention also encompasses a method for removing, depleting or inactivating MIF from blood, blood plasma, blood serum or other body fluids, in which said apheresis material or adsorbant is brought into contact with blood, blood plasma, blood serum or other body fluids of a patient extracorporally. If the apheresis material or adsorbant of the invention is in the form of particles or beads, then they are advantageously packed into a flow chamber or a column, through which the blood, blood plasma, blood serum or other body fluids of a patient is passed extracorporeally. Before or after a treatment in which MIF is depleted, one or more further treatment stages for the blood or other fluids can be carried out. Several treatments of the blood or other fluids can be carried out in successive units, in which MIF is depleted by adsorption, to achieve the desired end concentration of MIF, before the blood or other body fluid is reinfused into a patient.

### EXAMPLES

### Example 1

### Production of an Apheresis Material in Accordance with the Invention to Adsorb MIF from Blood Plasma

### 1.1. Production of Mercaptopyridine or Hexanethiol Acrylate Beads (No Spacer)

2 g of oxirane polyacrylate beads (Toyopearl™ HW70EC, Tosoh Biosep, Stuttgart) with a mean particle diameter of 140 μm, a mean exclusion threshold of 800 000 Da and a mean oxirane content of 4.0 mmol/g was reacted with 20 ml of 0.1M mercaptopyridine or 0.1 M hexane thiol in DMF for 24 h at 40° C. After completion of the reaction and washing several times with distilled water, the beads were dried at 40° C. in a vacuum drier.

### 1.2. Production of Thiophilic Acrylate Beads (No Spacer)

3 g of Toyopearl HW70EC beads were reacted in 20 ml of 4M sodium hydrogen sulfide solution (pH 11) for 1 h. After careful washing with distilled water, the beads were reacted with divinyl sulfone (0.4 M) in 20 ml of 0.1 M carbonate buffer (pH 11) at ambient temperature. The beads were then washed with distilled water and stirred for 45 min in 20 ml of a 2.3M mercaptoethanol solution in 0.5 M sodium carbonate (pH 11) at ambient temperature. Finally, the beads were washed to neutrality and dried in a vacuum drier.

1.3. Production of Acrylate Beads Modified with Mercaptopyridine or Hexanethiol, with Polyacrylate Spacer

5 g of Toyopearl HW70EC beads were aminated in 20 ml of 32% ammoniacal solution for 24 h at ambient temperature and then washed to remove the ammonia. The beads were then re-suspended in 45 ml of 0.1M NaOH and 0.6 g of 4,4′-azobis-(4-cyanopentanoic acid). After adding 0.85 g of N-hydroxysuccinimide and 0.85 g of 1-ethyl-3-(3-dimethylaminopropyl)-carbodiimide, the residue was stirred for 16 h at ambient temperature. After subsequent rinsing with water and 2-propanol, graft polymerization of the acrylate spacer was carried out by reacting the beads with 2.5 g of glycidyl methacrylate in 100 ml of 2-propanol. The reaction was carried out in a nitrogen atmosphere at 75° C. for 6 h with gentle stirring. After washing with propanol and water, mercaptopyridine was bound via the oxirane groups of the grafted polymer side chains. To this end, the beads were placed in 40 ml of 2-propanol and reacted for 24 h at 40° C. after adding mercaptopyridine (0.1M). It was then washed with propanol and water.

1.4. Production of Acrylate Beads Modified with the Catecholamines Dopamine or Norepinephrine, with Polyacrylate Spacer

5 g of Toyopearl HW70EC beads were aminated in 20 ml of 32% ammoniacal solution for 24 h at ambient temperature and then washed to remove the ammonia. The beads were then re-suspended in 45 ml of 0.1M NaOH and 0.6 g of 4,4′-azobis-(4-cyanopentanoic acid). After adding 0.85 g of N-hydroxysuccinimide and 0.85 g of 1-ethyl-3-(3-dimethylaminopropyl)-carbodiimide, the residue was stirred for 16 h at ambient temperature. After subsequent rinsing with water and 2-propanol, graft polymerization of the acrylate spacer was carried out by reacting the beads with 2.5 g of glycidyl methacrylate in 100 ml of 2-propanol. The reaction was carried out in a nitrogen atmosphere at 75° C. for 6 h with gentle stirring. Then the beads have been reacted with catecholamines according to the following schemes:

A: 0.75 g beads+0.95 g Dopamine in 10 ml sodium borate buffer (pH 10.5)
B: 1.5 g beads+0.5 g Norepinephrine in 10 ml sodium borate buffer (pH 10.5).

### Example 2

### Adsorption of MIF from PBS Buffer

The materials prepared in Examples 1.1. to 1.3. and S-hexyl-glutathione-agarose beads (Sigma-Aldrich, Munich) were tested for their ability to bind recombinant human MIF (rhuMIF) from PBS buffer (pH 7.2). The control was the base material without ligand modification. To carry out the tests, 1.2 ml of the beads was placed in columns which were equipped with a frit. It was then rinsed with PBS buffer and incubated for 15 min with an rhuMIF solution. After the incubation period, the solution was separated from the beads through the frit and the concentration of rhuMIF was determined using the Bradford protein quantification test (BioRad). The evaluation was carried out with the help of a bovine serum albumine (BSA) standard. Under the chosen conditions of adsorbing MIF from a PBS buffer solution, MIF quantification by a general protein assay is sufficient as no other interfering proteins are present. The binding capacities determined from the differences in the rhuMIF concentrations before and after incubation were respectively normalized to the bead mass. The results are shown in Tables 1 and 2.

TABLE 1
MIF binding capacity from PBS buffer in μg rhuMIF/ml beads

| | Adsorption capacity |
|---|---|
| Adsorbant | [μg/ml] |
| sepharose beads | 0.6 |
| acrylate beads | 0.9 |
| S-hexyl-glutathione-agarose beads | 2.4 |
| hexanethiol acrylate beads (no spacer) | 1.1 |
| mercaptopyridine acrylate beads (no spacer) | 1.5 |
| thiophilic acrylate beads (no spacer) | 1.6 |

(MIF starting concentration: 20 μg/ml),

TABLE 2
MIF binding capacity from PBS buffer, in μg rhuMIF/ml beads

| | Adsorption capacity |
|---|---|
| Adsorbant | [μg/ml] |
| S-hexyl-glutathione-agarose beads | 3.8 |
| mercaptopyridine acrylate beads (with | 6.3 |
| polymethacrylate spacer) | |

(MIF starting concentration: 50 μg/ml)

### Example 3

### Adsorption of MIF from Human Plasma

The materials prepared in Example 1.1. to 1.3. were tested in a batch process for their binding properties regarding rhuMIF from human plasma. To this end, 1.2 ml of beads were incubated with 500 μl of fresh ACD-anticoagulated human plasma for 15 min at ambient temperature. The human plasma was supplemented with 10 μg/ml of rhuMIF prior to incubation. Finally, prior to and after incubation, the MIF concentration was measured using a huMIF sandwich-ELISA (R&D Systems, MAB289 and BAF289). The binding capacities, which were determined from the MIF concentrations before and after incubation, are shown in Table 3.

TABLE 3
MIF binding capacity from human plasma, in μg rhuMIF/ml beads

| | Adsorption capacity |
|---|---|
| Adsorbant | [μg/ml] |
| S-hexyl-glutathione-agarose beads | 1.3 |
| mercaptopyridine acrylate beads (with | 2.6 |
| polymethacrylate spacer) | |

(MIF starting concentration: 10 μg/ml)

### Example 4

### Adsorption of MIF from Human Plasma

The materials of example 1.4 were rinsed with sodium borate buffer (pH 10.5) and reverse osmosis (RO) water. Then the adsorption capacity of the beads regarding rhuMIF from human plasma was determined. Therefore, 200 μl beads were incubated with freshly dotated human plasma for 15 minutes. Non-modified glycidyl methacrylate (GMA) grafted beads served as control. The plasma was spiked with 70 ng/ml rhuMIF prior to incubation and citrate was used as anticoagulant. The MIF concentration in the supernatant was measured pre and post incubation using a huMIF sandwich ELISA (MAB289 and BAF289, R&D Systems). The results are shown in Table 4 showing the MIF concentrations in the supernatants after the incubation experiment.

TABLE 4
rhuMIF concentration after incubation of MIF-spiked
human plasma (70 ng/ml) with adsorbants.

| | rhuMIF in supernatant |
|---|---|
| Adsorbant | [ng/ml] |
| Control | 52 |
| A: dopamin-acrylate | 2 |
| B: norepinephrine-acrylate | 17 |

### Example 5

### Investigations of MIF Binding Specificity

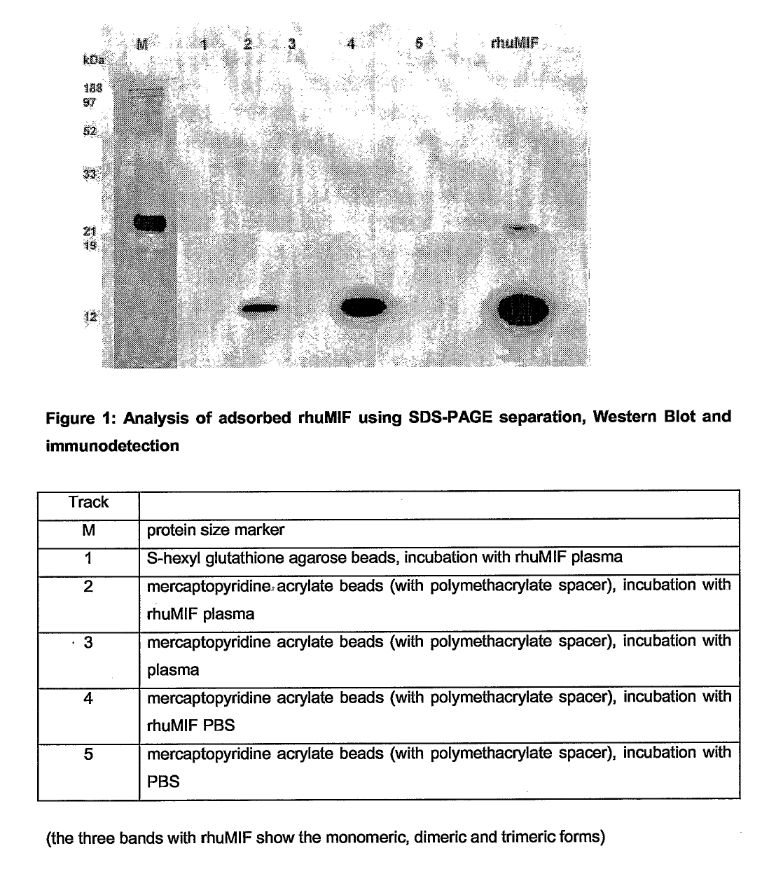

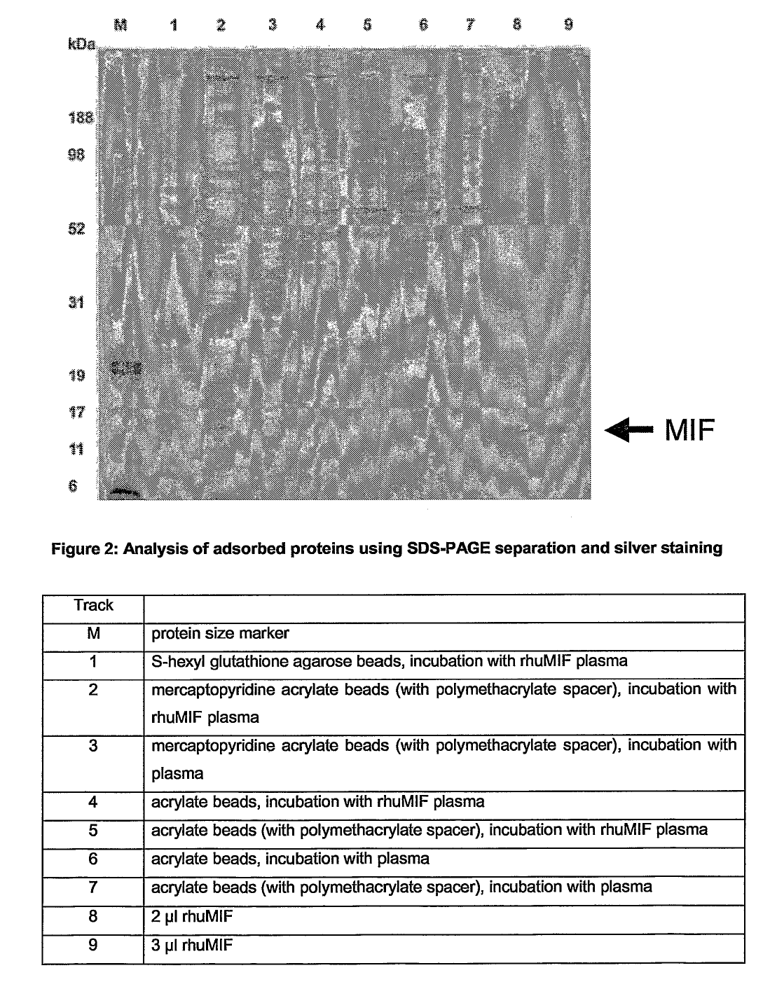

To investigate the specificity of MIF binding, following incubation, the materials from the binding tests were initially carefully washed with PBS buffer in various media. To desorb the adsorbed proteins, the adsorbants were then boiled for 7 min at 100° C. in SDS-containing loading buffer for SDS-PAGE (sodium dodecylsulfate polyacrylamide gel electrophoresis). The loading buffer had the following composition: 2.5 ml of 0.5 M Tris (pH 6.8), 4 ml of 10% SDS, 2 ml of glycerol, 1 ml of β-mercaptoethanol and 1 ml of bromophenol blue. In addition, the residue was separated by SDS-PAGE. To reveal the rhuMIF bands, the proteins were transferred onto a nitrocellulose membrane by Western Blot, the membrane was incubated with a mouse anti-huMIF antibody (MAB298 anti-huMIF, R&D Systems). After washing the membrane, it was incubated with anti-mouse antibody coupled to horseradish peroxidase (POD) and the Western Blot was stained in known manner. The Western Blot is shown in FIG. 1. An analysis of the adsorbed proteins was made by SDS PAGE separation of the material that was desorpted from the adsorbant and subsequent silver staining. The result is shown in FIG. 2.

1. An apheresis material or adsorbant for removing, depleting or inactivating MIF (macrophage migration inhibitory factor) from blood, blood plasma, blood serum or other body fluids, comprising a solid carrier material with MIF-binding molecules or functional groups immobilized on the surface of said solid carrier material.

2. An apheresis material or adsorbant according to claim 1, characterized in that the immobilized MIF-binding molecules or functional groups are bound to the carrier material via a spacer or polymer side chains grafted onto the carrier material.

3. An apheresis material or adsorbant according to one of the preceding claims, characterized in that the immobilized MIF-binding molecules or functional groups are bound to the carrier material via polyacrylate side chains grafted onto the carrier material.

4. An apheresis material or adsorbant according to one of the preceding claims, characterized in that the immobilized MIF-binding molecules or functional groups are selected from inhibitors of the catalytic and/or enzymatic activity of MIF.

5. An apheresis material or adsorbant according to one of the preceding claims, characterized in that the immobilized MIF-binding molecules or functional groups are selected from substrates or co-substrates for the catalytic and/or enzymatic activity of MIF.

6. An apheresis material or adsorbant according to one of the preceding claims, characterized in that the immobilized MIF-binding molecules or functional groups are selected from catecholamines or derivatives thereof, preferably selected from the group consisting of dopa, dopamine, norepinephrine (noradrenaline), epinephrine (adrenaline) or derivatives thereof.

7. An apheresis material or adsorbant according to one of the preceding claims, characterized in that the immobilized MIF-binding molecules or functional groups are dopamine.

8. An apheresis material or adsorbant according to one of the preceding claims, characterized in that the immobilized MIF-binding molecules or MIF-binding functional groups are selected from molecules containing mercapto groups or thiol groups.

9. An apheresis material or adsorbant according to one of the preceding claims, characterized in that the immobilized MIF-binding molecules are mercaptopyridine residues.

10. An apheresis material or adsorbant according to one of the preceding claims, characterized in that the carrier material is a porous material, preferably a membrane, a particle bed, a fiber mat or beads.

11. An apheresis material or adsorbant according to one of the preceding claims, characterized in that the carrier material is a biocompatible polymer material, preferably polyethersulfone (PES), polypropylene (PP), polysulfone (PSU), polymethylmethacrylate (PMMA), polycarbonate (PC), polyacrylonitrile (PAN), polyamide (PA), polytetrafluoroethylene (PTFE), cross-linked polystyrene-polyethylene glycol (PS-PEG), cyclo-olefin copolymer (COC), cellulose acetate (CA) or mixtures or copolymers thereof, with hydrophilized polymers such as polyvinylpyrrolidone (PVP) or polyethylene oxide (PEO).

12. An apheresis material or adsorbant according to one of the preceding claims, characterized in that the immobilized MIF-binding molecules or MIF-binding functional groups are selected from anti-MIF antibodies or fragments or derivatives thereof having at least one MIF-specific binding site.

13. An apheresis material or adsorbant according to one of the preceding claims, characterized in that the immobilized MIF-binding molecules or MIF-binding functional groups are selected from cellular MIF-binding proteins or domains or sequences thereof having at least one MIF-specific binding site.

14. An apheresis material or adsorbant according to one of the preceding claims, characterized in that the immobilized MIF-binding molecules or MIF-binding functional groups are JAB1/CSN5 or domains or sequences thereof having at least one MIF-specific binding site.

15. An apheresis material or adsorbant according to one of the preceding claims, characterized in that the immobilized MIF-binding molecules or MIF-binding functional groups are CD74 or domains or sequences thereof having at least one MIF-specific binding site.

16. An apheresis material or adsorbant according to one of the preceding claims, characterized in that the immobilized MIF-binding molecules or MIF-binding functional groups are MHC-II molecules or domains or sequences thereof having at least one MIF-specific binding site.

17. An apheresis material or adsorbant according to one of the preceding claims, characterized in that the immobilized MIF-binding molecules or MIF-binding functional groups are BNPL or domains or sequences thereof having at least one MIF-specific binding site.

18. An apheresis material or adsorbant according to one of the preceding claims, characterized in that the immobilized MIF-binding molecules or MIF-binding functional groups are myosin light chain kinase (MLCK) or domains or sequences thereof having at least one MIF-specific binding site.

19. A method for removing, depleting or inactivating MIF (macrophage migration inhibitory factor) in blood, blood plasma, blood serum or other body fluids, in which the apheresis material or adsorbant according to one of claims 1 to 15 is brought into extracorporeal contact with the blood, blood plasma, blood serum or other body fluids of a patient.

20. Use of an apheresis material or adsorbant according to one of claims 1 to 15, for extracorporeal removal of or depletion of MIF (macrophage migration inhibitory factor) in a patient's blood, blood plasma, blood serum or other body fluids of a patient.


SEQUENCE LISTING (1 sequence)

SEQ ID NO: 1 - protein, 114 aa, Human
PMFIVNTNVPRASVPDGFLSELTQQLAQATGKPPQYIAVHVVPDQLMAFGGSSEPCALCSLHSIGKIGGAQNRSYSKLLCGLLAERLRISPDRVYINYYDMNAANVGWNNSTFA